(19) United States
(12) Patent Application Publication
(10) Pub. No.: US 2008/0187910 A1
(43) Pub. Date: Aug. 7, 2008

(54) Nucleic acid assay and method of detecting the presence of a nucleic acid sequence

(76) Inventor: Thor W. Nilsen, Stirling, NJ (US)

Correspondence Address: WATOV & KIPNES, P.C., P.O. Box 247, Princeton Junction, NJ, 08550, US

(21) Appl. No.: 10/607,188

(22) Filed: Jun. 26, 2003

Related U.S. Application Data
(60) Provisional application No. 60/392,700, filed on Jun. 26, 2002.

(51) Int. Cl. C12Q 1/68; C12M 1/00

(52) U.S. Cl. 435/6; 435/287.2

(57) ABSTRACT

The present invention is directed to an assay and a method of detecting the presence of a specific sample nucleic acid sequence in a sample suspected of containing the same. The assay utilizes nucleic acid matrices to facilitate reactions which detect the presence of a nucleic acid sequence through formation of a hybridized or bound nucleic acid structure having a portion capable of disassociating therefrom through the action of a structure specific cleaving enzyme.

### RELATED APPLICATION

The present Application claims the priority of U.S. Provisional Application Ser. No. 60/392,700, filed Jun. 26, 2002, and entitled “Signalling and Reporting Using Dendrimer Technology”, the content of which is fully incorporated herein by reference.

### FIELD OF THE INVENTION

The present invention relates to nucleic acid assays, and more particularly to a nucleic acid assay and a method of detecting the presence of a nucleic acid sequence through formation of a hybridized or bound nucleic acid structure having a portion capable of disassociating therefrom through the action of a disassociating agent.

### BACKGROUND OF THE INVENTION

Changes in gene expression patterns or in a DNA sequence can have profound effects on biological functions. Such variations in gene expression may result in altered physiological and pathological processes. Similarly, there are many applications which require the determination of the presence and absence of specific nucleic acid sequences. Developing DNA technologies are providing rapid and cost-effective methods for determining the presence or absence of nucleic acid sequences, and for identifying gene expression and genetic variations, each on a large-scale level. One useful development is the DNA microarray useful for rapidly detecting and assaying samples of target nucleic acid. Each microarray is capable of performing the equivalent of thousands of individual “test tube” experiments over a short time period thereby providing rapid detection of thousands of expressed genes. Microarrays have been implemented in a range of applications such as analyzing a sample for the presence of nucleic acid sequences, gene variations or mutations (i.e. genotyping), or for patterns of gene expression.

Generally, a microarray comprises a substantially planar substrate such as a glass cover slide, a silicon plate or nylon membrane, coated with a grid of tiny spots or features of about 20 microns in diameter. Each spot or feature contains millions of copies of a specific sequence of nucleic acid extracted from a strand of a pair bonding molecule having characteristics of a nucleic acid such as DNA or RNA. Examples of such nucleic acid like polymers include Eragen's extended code, fluorinated bases, 2′-methoxy bases, and the like, each of which exhibit polymer recognition properties. Due to the number of features involved, a computer is typically used to keep track of each sequence located at each predetermined feature. Messenger RNA (mRNA) is extracted from a sample of cells. The mRNA serving as a template, is reverse transcribed to yield a complementary DNA (cDNA). As a first example of the prior art techniques, one or more labels or markers such as fluorescent compounds are directly incorporated into the copies of cDNA during the reverse transcription process. The labeled copies of cDNA are broken up into short fragments and washed over the microarray. Under suitable hybridization conditions, the labeled fragments are hybridized or coupled with complementary nucleic acid sequences (i.e. gene probes) attached to the features of the microarray for ready detection thereof. This labeling method has been commonly referred to as “direct incorporation”.

Upon hybridization of the cDNA to the microarray, a detectable signal (e.g. fluorescence) is emitted for a positive outcome from each feature containing a cDNA fragment hybridized with a complementary gene probe attached thereto. The detectable signal is visible to an appropriate sensor device or microscope, and may then be detected by the computer or user to generate a hybridization pattern. Since the nucleic acid sequence at each feature is known, any positive outcome (i.e. signal generation) at a particular feature indicates the presence of the complementary cDNA sequence in the sample cell. Although there are occasional mismatches, the attachment of millions of gene probes at each spot or feature ensures that the detectable signal is strongly emitted only if the complementary cDNA of the test sample is present.

It is known that the above-described prior art method requires significant time, effort and labor in the preparation and assay of the sample.

Accordingly, it would be highly desirable to substantially reduce the amount of time and the number of steps required for preparing a sample and performing the assay without sacrificing desirable attributes such as sensitivity, low background “noise”, and minimal “false positives”. It would also be desirable to design an assay which can be carried out directly on cell lysates utilizing minimal nucleic acid material and significantly minimizing erroneous signal production typically associated with procedures that require polymerase chain reaction processing. It would be a significant advance in the art of detecting and identifying nucleic acid sequences to further provide a nucleic acid assay that significantly reduces the complexity and the labor needed to prepare the nucleic acid samples and conduct the assay which can be carried out using conventional laboratory reagents, equipment and techniques.

### SUMMARY OF THE INVENTION

The present invention relates generally to nucleic assays for detecting the presence of a specific sequence of nucleic acid in a sample through the process of hybridization. The present invention is capable of identifying nucleic acid sequences through formation of specific nucleic acid hybridization structures typically in the form of nucleic acid matrices having portions which specifically interact with target molecules (e.g., sample nucleic acid sequences) via disassociation or cleavage reaction. Such a disassociation reaction can be initiated by a disassociation agent at a predetermined location whereby the resulting cleavage or disassociation indicates the presence of specific nucleic acid sequences. The present invention significantly reduces the time and labor that are typically required to process and detect the sample nucleic acid to obtain information about the sequences of the sample nucleic acid and the source from which it was obtained.

The nucleic acid assay of the present invention is further designed to reduce the steps and the time for determining the presence of at least one specific nucleic acid sequence in a sample nucleic acid obtained from a biological sample.

The present invention relies on a structure specific cleavage reaction that achieves enhanced nucleic acid discrimination with a disassociating agent which recognizes a specific structure which forms only upon the presence of a positive response. The specific structure interacts with the disassociating agent to initiate a signal amplification process and/or a signal generating response. This reaction can be implemented in a simple and rapid manner without the need for prior polymerase chain reaction amplification of the sample nucleic acid.

In one aspect of the present invention, there is provided a nucleic acid assay for detecting the presence of a specific sample nucleic acid sequence in a sample suspected of the containing the same where the nucleic acid assay comprises:

(a) a matrix comprising at least one first site for receiving an invader oligonucleotide and at least one second site for receiving a probe oligonucleotide;

(b) at least one invader oligonucleotide for attaching to the first site of the matrix, said invader oligonucleotide having an invader nucleic acid sequence for binding to a first portion of the sample nucleic acid sequence;

(c) at least one probe oligonucleotide for attaching to the second site of the matrix, said probe oligonucleotide having a first probe nucleotide portion for binding to a second portion of the sample nucleic acid sequence and a second probe nucleotide portion which does not bind to the sample nucleic acid sequence;

(d) a first disassociating agent for disassociating the second probe nucleotide portion of the probe oligonucleotide from the first probe nucleotide portion upon the concurrent binding of the invader nucleic acid sequence of an invader oligonucleotide to the first portion of the sample nucleic acid sequence and the first probe nucleotide portion for binding to the second portion of the sample nucleic acid sequence; and

(e) detection means for detecting the degree to which the second probe nucleotide portion of the probe oligonucleotide has disassociated from the first probe nucleotide portion thereof.

In one preferred embodiment of the present invention, the matrix is in the form of a nucleic acid dendrimer.

In one particular embodiment of the present invention, the present invention may be implemented in the form of a microarray with excellent sensitivity and low background “noise”, and minimal “false positives”. The method of the present invention may be used in a range of high-throughput nucleic acid applications such as quantifying nucleic acid sequences, profiling gene expression and analyzing functional nucleic acid.

In another particular aspect of the present invention, there is provided a method of detecting the presence of a specific sample nucleic acid sequence in a sample suspected of containing the same. The method comprises the steps of:

contacting the sample with a matrix comprising at least one first site with an invader oligonucleotide having an invader nucleic acid sequence for binding to a first portion of the specific sample nucleic acid sequence, and at least one second site with a probe oligonucleotide having a first probe nucleotide portion for binding to a second portion of the specific sample nucleic acid sequence and a second probe nucleotide portion which does not bind to the specific sample nucleic acid sequence to yield a sample-matrix mixture;

treating the sample-matrix mixture at a temperature and for a time sufficient to induce the invader oligonucleotide to bind to the first portion of the specific sample nucleic acid sequence and to induce the first probe nucleotide portion of the probe oligonucleotide to bind to the second portion of the specific sample nucleic acid sequence to yield a hybridization complex;

contacting the hybridization complex with a disassociating agent wherein the disassociating agent disassociates the second nucleotide portion from the first probe nucleotide portion thereof; and

detecting the degree to which the second probe nucleotide portion of the probe oligonucleotide has disassociated from the first probe nucleotide portion thereof.

### DETAILED DESCRIPTION OF THE INVENTION

The present invention is generally directed to a nucleic acid assay and a method designed in a manner that provides a low-cost, simplified and reliable system for implementing one or more simultaneous analysis of nucleic acid sample material within a sample. The present invention utilizes invasive cleavage assay technology to obviate the need for target amplification and additional enzymatic signal enhancement, and can detect relatively low levels of nucleic acid in a short period of time. The present invention is based on a structure specific cleavage reaction that achieves enhanced nucleic acid discrimination through the use of a disassociating agent such as, for example, a 5′-nuclease which recognizes a three component substrate or structure formed upon hybridization of target or sample nucleic acid with a probe oligonucleotide (e.g., a labeled nucleic acid) and upstream oligonucleotides. The assay of the present invention is simple and time efficient to implement and comprises a cleavage reaction which generates a detectable signal (e.g., fluorescence) on surfaces of one or more nucleic acid matrices as will be further described hereinafter.

The present invention utilizes a disassociating agent which becomes active when two independent bindings or hybridizations occur simultaneously on a nucleic acid sequence and an unbound portion extending therefrom to form a high-fidelity nucleic acid structure. The present invention is further directed to a method for implementing the assay of the present invention.

The present invention further provides the advantage of preparing the sample nucleic acid in shorter period of time, using fewer steps but providing the sensitivity, low background “noise”, and minimal “false positives” required for laboratory and clinical use. The cost effective and efficient manner by which the sample nucleic acid sequence is prepared and by which the method of the present invention can be implemented using conventional laboratory techniques, equipment and reagents, makes the present invention especially suitable for use in nucleic acid detection applications such as gene expression profiling and high-throughput functional nucleic acid analysis. The term “sample nucleic acid” as used herein is meant to encompass any DNA or RNA-based genetic material processed or extracted from a natural source for assaying.

Before the present invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

The terms “matrix” or “nucleic acid matrix” each refer generally to a collection of at least three single-stranded nucleic acid molecules held in close association in a dimensionally ordered arrangement via intermolecular base pairing and/or covalent cross links. The nucleic acid matrix generally includes at least one single stranded nucleic acid end on which any functional group may be attached thereto via covalent or non-covalent bonding. Such bonding may be implemented by any bonding chemistries including, but not limited to, psoralen cross linking and ligation. An example of a suitable matrix for use in the present invention is 3DNA® obtained from Genisphere Inc. of Hatfield, Pa.

Although the following detailed description is made in reference to the embodiments of the present invention related to nucleic acids, it will be appreciated by those skilled in the art that this is merely a representative embodiment, the spirit and scope of the invention encompasses any pair bonding molecules or polymers when nucleic acid is referred to. The use of pair bonding molecules such as nucleic acid to build dimensionally ordered matrices for implementation with the assay and method of the present invention is fundamental to this invention. The purification of single stranded base pairable molecules capable of partial annealing, generating matrix building units from the molecules, followed by the assembly of the matrix building units into an ordered matrix either in solution or on a solid support is known in the art.

The term “label” is used herein in a broad sense to refer to agents that are capable of generating a detectable signal, either directly or through interaction with one or more additional members of a signal producing system including, for example, dye and quencher-based systems. Labels that are directly detectable and may find use in the present invention include, for example, fluorescent labels such as fluorescein, rhodamine, BODIPY, cyanine dyes (e.g. from Amersham Pharmacia), Alexa dyes (e.g. from Molecular Probes, Inc.), fluorescent dye phosphoramidites, Cy3®, Cy5®, and the like; and radioactive isotopes, such as ³²S, ³²P, ³ H, etc.; and the like.

The assay of the present invention as stated previously above relies on a reaction referred to as invasive cleavage reaction that is carried out by cleavage of a specific and unique structure. This unique structure is generally formed by hybridization of a target or sample nucleic acid sequence to upstream oligonucleotides referred herein as invader oligonucleotide and a probe oligonucleotide (i.e., signal generating and amplification component) that overlap by one base. The unpaired region at the 5′-end of the probe oligonucleotide forms a “flap” or “flap sequence” that is a substrate for cleavage by a structure specific disassociating agent (e.g., 5′-nucleoase). The flap is cleaved off by the disassociating agent to trigger a signal generating event (e.g., quencher-dye label system) or a signal cascade process, thus indicating a positive response. In the present invention, the probe oligonucleotide and the invader oligonucleotide are attached to nucleic acid matrices to implement the invasive cleavage reaction on a solid support.

The selection of the probe oligonucleotide and invader oligonucletide may be made depending on the type and amount of sample nucleic acid available, the signal generating system used, the degree of stability desired, the time requirements, the hybridization conditions and the like. The selection and attachment of the probe oligonucleotides and invader oligonucleotides to nucleic acid matrices such as nucleic acid dendrimers can be readily be implemented by skilled artisans. For example, the attachment of the probe and invader oligonucleotides can be accomplished through nucleic acid ligation reactions as known by those of ordinary skill in the art.

In one embodiment of the present invention, the assay may be in the form of a single nucleic acid matrix or an array of nucleic acid matrices which form a solid support for the components of the invasive cleavage reaction. Each nucleic acid matrix may be fixed or stably associated with the surface of a support substrate (i.e., microtiter plates or biochip) is prepared as conventionally known in the art. A variety of different support substrates that may be used are known in the art. The support substrates with which the nucleic acid matrices are stably associated may be fabricated from a variety of materials, including plastic, ceramic, metal, gel, membrane, glass, and the like. A preferred support substrate surface is one that is transparent to the excitation and emission wavelengths of a signal generated by a label moiety.

The support substrates and array of nucleic acid matrices may be produced according to any convenient methodology, such as pre-forming the nucleic acid matrices and then stably associating them with the surface of the support through covalent or non-covalent attachments. A preferred attachment is a covalent attachment implemented via cross linking of the nucleic acid matrices to oligonucleotides covalently bound to the surface via amino link, phosphorate or other chemical moieties. It will be understood that the present invention may be implemented through a number of different array configurations and methods for their production are known to those of skill in the art.

Examples of a suitable nucleic acid matrix may be selected from dentritic nucleic acid molecules or dendrimers, hyperbranched architecture molecules, regular lattice molecules and the like, each of which are capable of retaining at least one functional group (i.e., 5′-probe oligonucleotide and 3′-invader oligonucleotide) in an aqueous phase.

One preferred example of a nucleic acid matrix is a “dendritic nucleic acid molecule”, or “dendrimer”. Dendrimers are complex, highly branched molecules, and are comprised of a plurality of interconnected natural or synthetic monomeric subunits of double-stranded DNA forming into stable spherical-like core structures with a predetermined number of “free ends” or “arms” extending therefrom. Typically, the nucleic acid matrix will have multiple, typically many first and second arms. Dendrimers provide efficient means for facilitating the invasive cleavage reactions as will be described hereinafter. Dendrimers will be described hereinafter as illustrative of a suitable nucleic acid matrix.

Each dendrimer generally includes two types of hybridization “free ends” or “arms” extending from the core surface. Each dendrimer may be configured to include at least one hundred arms of each type. The arms are each composed of a single-stranded DNA of a specific sequence that can be ligated to a functional molecule such as such as an invader oligonucleotide and a probe oligonucleotide as will be further described hereinafter. The functional molecule can be attached to one type of arm to provide the dendrimer with the specific reaction capabilities required to perform or implement the assay of the present invention.

Using simple DNA labeling, hybridization, and ligation reactions, a dendrimer can thus be configured to act as a highly labeled, target specific substrate or matrix, and therefore may be used in a microarray system for implementing nucleic acid analysis including gene expression analysis, a protocol of which is shown in Examples 1 and 2 herein. Dendrimers are described in greater detail in U.S. Pat. Nos. 5,175,270 and 5,484,904, in Nilsen et al., Dendritic Nucleic Acid Structures, J. Theor. Biol., 187, 273-284 (1997); and in Stears et al., A Novel, Sensitive Detection System for High-Density Microarrays Using Dendrimer Technology, Physiol. Genomics, 3:93-99 (2000), the entire content of each are incorporated herein by reference. Further information regarding the structure, configuration and production of dendrimers including protocols for attach functional molecules to the arms of the dendrimers may also be found in U.S. Pat. Nos. 5,175,270, 5,484,904, and 5,487,973, the contents of each are incorporated herein by reference.

In a further preferred embodiment of the present invention, the dendrimer is composed of at least one first arm each comprising an invader oligonucleotide having a nucleic acid sequence complementary to a first nucleic acid portion of a sample nucleic acid and at least one second arm each comprising a probe oligonucleotide. The probe oligonucleotide includes a specific, target complementary sequence which is fixedly attached to the second arm of the matrix, and is capable of hybridizing or binding with a second nucleic acid portion of the sample nucleic acid. The binding of the invader and probe oligonucleotide with the respective first and second nucleic acid portions of the sample nucleic acid forms an invader and probe oligonucleotides-sample complex or a hybridized complex.

The probe oligonucleotide further includes a generic flap sequence which forms the 5′ end of the oligonucleotide and which is non-complementary to the sample nucleic acid. A disassociating agent is added in the presence of the hybridization reaction. Upon formation of the invader and probe oligonucleotides-sample complex, the disassociating agent recognizes and releases the flap sequence, which generates an event. The probe oligonucleotide is preferably tailored to generate a detectable signal upon the release of the flap sequence.

The disassociating agent may be selected from a Lewis base, an acid, chaperoning, enzymes such as a nuclease, a restriction enzyme, a recombinase, a ligase, a transferase and a phosphatase, and the like and combinations thereof. An example of a suitable disassociating agent is enzymatic compound marketed under the tradename CLEAVASE® which is available from Third Wave Technologies, Inc. of Madison, Wis.

Once the flap sequence is released, the cleaved probe oligonucleotide bound to the sample nucleic acid is released which allows a new probe oligonucleotide to bind to the sample nucleic acid. The disassociating agent thereafter releases the flap sequence from the newly bound probe oligonucleotide and the process is repeated. Each of the flap sequence released can be detected through suitable means such as a signal generating label attached to each of the probe oligonucleotides. Each sample nucleic acid may induce the release of several thousand flap sequences per hour to produce an effective signal amplifier. Accordingly, a single sample nucleic acid can generate a detectable event as a result of the accumulation of the released flap sequences and corresponding signals generated.

In implementing the assay of the present invention, a sample nucleic acid is contacted with the nucleic acid matrix ligated with a plurality of invader oligonucleotide and probe nucleotide at an optimal reaction temperature, typically from about 60° to 65° C. Each of the invader oligonucleotides binds and remains annealed to the sample nucleic acid, while the probe oligonucleotides each undergo rapid dissolution and reassociation with the sample nucleic acid.

In the presence of a first disassociating agent (e.g., 5′-nuclease), the 5′-flap sequence is cleaved from the probe oligonucleotides associated with each of the invader and probe oligonucleotides-sample complex. This results in a target specific accumulation of the generic 5′-flap sequence. The probe oligonucleotide may be optionally prepared with a label which generates a detectable signal upon the release of the generic 5′-flap sequence. The signal may be generated by the flap sequence or the portion of the probe oligonucleotide complementary to the sample nucleic acid. Optionally, the signal generated by the probe oligonucleotide may be further enhanced by adding arrestor oligonucleotides which can bind with any uncleaved probe oligonucleotides that may be present.

Optionally, to enhance signal gain, a template oligonucleotide or secondary reaction substrate having a first portion complementary to the 5′-flap sequence and a second portion complementary to at least a portion of a reporter oligonucleotide, may be added to the presence of the released 5′-flap sequences. A reporter oligonucleotide including a flap sequence having a quencher and a binding sequence having a dye capable of generating a detectable signal when flap sequence and quencher is released, is also added to the presence of the released 5′-flap sequences and the template oligonucleotide. The reporter oligonucleotide binds to the template oligonucleotide after the 5′-flap sequence is bound to the template oligonucleotide. In the presence of a second disassociating agent (e.g., 5′-nuclease), the flap sequence of the reporter oligonucleotide is released therefrom when bound to the released 5′-flap sequence/template oligonucleotide. Similarly to the cleaved probe oligonucleotide described above, a signal is generated for detection.

Specific detection of each sample nucleic acid may be accomplished by using spectrally distinct fluorophores. The probe and/or reporter oligonucleotides preferably selected from fluorescence resonance energy transfer (FRET)-based oligonucleotides. More preferred fluorescence is reversed FRET wherein a quencher is attached to the flap sequence of the probe oligonucleotide and/or the flap sequence of the reporter oligonucleotide, and a fluorescent label or dye is attached to the binding portion of the probe and/or reporter oligonucleotide. In one example, the probe and/or reporter oligonucleotides each contain a fluroescein and a dabcyl quencher moiety. Thus, cleavage of the flap sequence of the probe/reporter oligonucleotide releases the quencher and commences signal generation.

One preferred form of fluorescence is fluorescence polarization which provides fluorescent molecules which when bound to the probe and/or reporter oligonucleotide and exposed to polarized light emit a specific degree of polarized light. However, when the fluorescent molecules are released from the corresponding oligonucleotide, they emit unpolarized light. This change in light emitted can be detected and equated to the amount of released fluorescent molecules.

Following hybridization and disassociation, a resultant hybridization pattern generated by the label may be detected through a suitable commercially available microarray scanners, radiological detectors, optical detectors, fluorescent microscopes or any existing event detection system. In detecting or visualizing the hybridization pattern, the intensity or signal value of the label may be qualitatively and/or quantitatively detected. The resultant pattern of the emitted signal of the assay may be visualized or detected in a variety of ways, with the particular manner of detection being chosen based on the particular label used in the present invention, where representative detection systems include scintillation counting, autoradiography, fluorescence measurement, calorimetric measurement, light emission measurement and the like.

Following detection or visualization, the signal pattern can be used to determine qualitative and/or quantitative information about the presence or absence of a specific nucleic acid of the sample, as well as the physiological source from which the sample nucleic acid was derived, for example. From this data, one can also derive information such as the quantity and types of nucleic acid sequences. Where one uses the subject methods in comparing sample nucleic acid from two or more physiological sources, the hybridization patterns may be compared to identify differences between the patterns. For certain applications with microarrays wherein each of the different nucleic acid matrices corresponds to a known gene is employed, any discrepancies can be related to a differential expression of a particular gene in the physiological sources being compared. Thus, the subject methods find use in differential gene expression assays, where one may use the subject methods in the differential expression analysis of: diseased and normal tissue, e.g. neoplastic and normal tissue; different tissue or subtissue types; and the like.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

### EXAMPLE 1

Preparation of a Nucleic Acid Dendrimer via Ligation Reactions

40 μg of core dendrimer (3DNA® obtained from Genisphere Inc. of Hatfield, Pa.) in 75.9 μl of reagent grade deionized and distilled water (RGDD), 20 μg of a first bridge oligonucleotide in 42.2 μl RGDD, 20 μg of a probe oligonucleotide in 40.0 μl RGDD, 20 μg of a second bridge oligonucleotide in 34.1 μl RGDD, 20 μg of an invader oligonucleotide in 34.5 μl RGDD and 1 μg of a probe biotin oligonucleotide in 5.8 μl RGDD were mixed together in a 1000 ml beaker. 127.5 μl RGDD and 40.0 μl of 10× ligation buffer was added to the beaker to yield a final volume of 400 μl as the reaction mixture.

The reaction mixture was heated to 50° C. and gradually cooled to 4° C. 700 ml RGDD were added to the beaker. 50 units of T4 DNA ligase were added to the beaker. The resulting mixture was incubated overnight at a temperature of about 14° C. to form a reagent for use in an assay of the present invention.

### EXAMPLE 2

### Preparation of a Nucleic Acid Dendrimer via Ligation Reactions

40 μg of core dendrimer (3DNA® obtained from Genisphere Inc.) in 75.9 μl of reagent grade deionized and distilled water (RGDD), 20 μg of a first bridge oligonucleotide in 42.2 μl RGDD, 20 μg of a probe oligonucleotide in 40.0 μl RGDD, 20 μg of a second bridge oligonucleotide in 34.1 μl RGDD, 20 μg of an invader oligonucleotide in 34.5 μl RGDD and 1 μg of a probe biotin oligonucleotide in 1.6 μl RGDD were mixed together in a 1000 ml beaker. 131.6 μl RGDD and 40.0 μl of 10× ligation buffer were added to the beaker to yield a final volume of 400 μl as the reaction mixture.

The reaction mixture was heated to 50° C. and gradually cooled to 4° C. 700 ml RGDD were added to the beaker. 50 units of T4 DNA ligase were added to the beaker. The resulting mixture was incubated overnight at a temperature of about 14° C. to form a reagent useful in an assay of the present invention.

What is claimed is:

1. A nucleic acid assay for detecting the presence of a specific sample nucleic acid sequence in a sample suspected of the containing the same, said nucleic acid assay comprising:
(a) a matrix comprising at least one first site for receiving an invader oligonucleotide and at least one second site for receiving a probe oligonucleotide;
(b) at least one invader oligonucleotide for attaching to the first site of the matrix, said invader oligonucleotide having an invader nucleic acid sequence for binding to a first portion of the sample nucleic acid sequence;
(c) at least one probe oligonucleotide for attaching to the second site of the matrix, said probe oligonucleotide having a first probe nucleotide portion for binding to a second portion of the sample nucleic acid sequence and a second probe nucleotide portion which does not bind to the sample nucleic acid sequence;
(d) a first disassociating agent for disassociating the second probe nucleotide portion of the probe oligonucleotide from the first probe nucleotide portion upon the concurrent binding of the invader nucleic acid sequence of an invader oligonucleotide to the first portion of the sample nucleic acid sequence and the first probe nucleotide portion for binding to the second portion of the sample nucleic acid sequence; and
(e) detection means for detecting the degree to which the second probe nucleotide portion of the probe oligonucleotide has disassociated from the first probe nucleotide portion thereof.

2. The nucleic acid assay of claim 1 wherein the probe oligonucleotide includes a first label for generating a detectable signal upon the disassociation of the second probe nucleotide portion from the first probe nucleotide portion thereof.

3. The nucleic acid assay of claim 2 wherein the first label of the probe oligonucleotide is selected from the group consisting of fluorescent dyes, fluorescein, rhodamine, cyanine dyes, Alexa dyes, fluorescent dye phosphoramidites, radioactive isotopes and combinations thereof.

4. The nucleic acid assay of claim 2 wherein the first label is operatively associated with the first probe nucleotide portion of the probe oligonucleotide.

5. The nucleic acid assay of claim 4 wherein the first label is attached to the first probe nucleotide portion of the probe oligonucleotide in operative association with a quencher attached to the second probe nucleotide portion of the probe oligonucleotide.

6. The nucleic acid assay of claim 2 wherein the first label is operatively associated with the second probe nucleotide portion of the probe oligonucleotide.

7. The nucleic acid assay of claim 6 wherein the first label is attached to the second probe nucleotide portion of the probe oligonucleotide in operative association with a quencher attached to the first probe nucleotide portion of the probe oligonucleotide.

8. The nucleic acid assay of claim 1 wherein the disassociating agent is selected from the group consisting of a Lewis base, a Lewis acid, a nuclease, a restriction enzyme, a recombinase, a ligase, a transferase, a polymerase, a phosphatase, a chaperonin and combinations thereof.

9. The nucleic acid assay of claim 1 further comprising:
at least one secondary reaction substrate having binding sites for receiving the disassociated second probe nucleotide portion of the probe oligonucleotide;
at least one reporter oligonucleotide having a first reporter nucleotide portion for binding to a portion of the secondary reaction substrate and a second probe nucleotide portion which does not bind to the secondary reaction substrate; and
a second disassociating agent for disassociating a second reporter nucleotide portion of the reporter oligonucleotide from the first reporter nucleotide portion upon the concurrent binding of the disassociated second probe nucleotide portion of the probe oligonucleotide to a binding site of the secondary reaction substrate and the first reporter nucleotide portion to a portion of the secondary reaction substrate.

10. The nucleic acid assay of claim 9 wherein the reporter oligonucleotide includes a second label for generating a detectable signal upon the disassociation of the second reporter nucleotide portion from the first reporter nucleotide portion thereof.

11. The nucleic acid assay of claim 10 wherein the second label of the reporter oligonucleotide is selected from the group consisting of fluorescent dyes, fluorescein, rhodamine, cyanine dyes, Alexa dyes, fluorescent dye phosphoramidites, radioactive isotopes and combinations thereof.

12. The nucleic acid assay of claim 10 wherein the second label is operatively associated with the first reporter nucleotide portion of the reporter oligonucleotide.

13. The nucleic acid assay of claim 12 wherein the second label is attached to the first reporter nucleotide portion of the reporter oligonucleotide in operative association with a quencher attached to the second reporter nucleotide portion of the reporter oligonucleotide.

14. The nucleic acid assay of claim 10 wherein the second label is operatively associated with the second reporter nucleotide portion of the reporter oligonucleotide.

15. The nucleic acid assay of claim 14 wherein the second label is attached to the second reporter nucleotide portion of the reporter oligonucleotide in operative association with a quencher attached to the first reporter nucleotide portion of the reporter oligonucleotide.

16. The nucleic acid assay of claim 9 wherein the first and second disassociating agents are the same.

17. The nucleic acid assay of claim 9 wherein the detection means is further capable of detecting the degree to which the second reporter nucleotide portion of the reporter oligonucleotide has disassociated from the first reporter nucleotide portion thereof.

18. The nucleic acid assay of claim 1 wherein the matrix comprises at least three single-stranded nucleic acid molecules held in close association in a dimensionally ordered arrangement.

19. The nucleic assay of claim 1 wherein the matrix is a member selected from the group consisting of nucleic acid dendrimer, hyperbranched architecture molecules, regular lattice molecules, and combinations of thereof.

20. The nucleic assay of claim 19 wherein the matrix is a nucleic acid dendrimer.

21. The nucleic acid assay of claim 1 wherein the matrix is affixed to a support substrate.

22. A method of detecting the presence of a specific sample nucleic acid sequence in a sample suspected of containing the same, said method comprising the steps of:
contacting the sample with a matrix comprising at least one first site with an invader oligonucleotide having an invader nucleic acid sequence for binding to a first portion of the specific sample nucleic acid sequence, and at least one second site with a probe oligonucleotide having a first probe nucleotide portion for binding to a second portion of the specific sample nucleic acid sequence and a second probe nucleotide portion which does not bind to the specific sample nucleic acid sequence to yield a sample-matrix mixture;
treating the sample-matrix mixture at a temperature and for a time sufficient to induce the invader oligonucleotide to bind to the first portion of the specific sample nucleic acid sequence and to induce the first probe nucleotide portion of the probe oligonucleotide to bind to the second portion of the specific sample nucleic acid sequence to yield a hybridization complex;
contacting the hybridization complex with a disassociating agent wherein the disassociating agent disassociates the second probe nucleotide portion from the first probe nucleotide portion thereof; and
detecting the degree to which the second probe nucleotide portion of the probe oligonucleotide has disassociated from the first probe nucleotide portion thereof.

23. The method of claim 22 wherein the probe oligonucleotide includes a label for generating a detectable signal upon the disassociation of the second probe nucleotide portion from the first probe nucleotide portion thereof.

24. The method of claim 23 wherein the label is operatively associated with the first probe nucleotide portion of the probe oligonucleotide.

25. The method of claim 24 wherein the label is attached to the first probe nucleotide portion of the probe oligonucleotide in operative association with a quencher attached to the second probe nucleotide portion of the probe oligonucleotide.

26. The method of claim 23 wherein the label is operatively associated with the second probe nucleotide portion of the probe oligonucleotide.

27. The method of claim 26 wherein the label is attached to the first probe nucleotide portion of the probe oligonucleotide in operative association with a quencher attached to the first probe nucleotide portion of the probe oligonucleotide.
